(19) United States
(12) Patent Application Publication
(10) Pub. No.: US 2010/0210975 A1
(43) Pub. Date: Aug. 19, 2010

(54) MULTI-STATE PERFORMANCE MONITORING SYSTEM

(75) Inventors: John J. Anthony, III, Norwich, CT (US); Scott E. Chalfant, Andover, CT (US)

Correspondence Address: MCCORMICK, PAULDING & HUBER LLP, CITY PLACE II, 185 ASYLUM STREET, HARTFORD, CT, 06103, US

(73) Assignee: SwimSense, LLC, Norwich, CT (US)

(21) Appl. No.: 12/691,414

(22) Filed: Jan. 21, 2010

Related U.S. Application Data
(60) Provisional application No. 61/146,292, filed on Jan. 21, 2009.
(60) Provisional application No. 61/183,571, filed on Jun. 3, 2009.

(51) Int. Cl. A61B 5/11

(52) U.S. Cl. 600/595

(57) ABSTRACT

The present invention is directed to systems and methods for monitoring performance of a variety of activities, preferably including swimming, in a number of states. The invention may comprise an analytics application, a primary device and, optionally, one or more secondary devices or third-party devices for sensing characteristics of the performance of an activity or the environment in which it is performed. The devices are worn during the performance of the activity and contain sensors, preferably accelerometers, for sensing information pertaining to the performance of one or more activities. The information sensed may be provided to a user in the form of real-time feedback and/or made available for long-term analytics and tracking.

### CROSS-REFERENCE TO RELATED APPLICATIONS

This application claims priority to U.S. Patent Application No. 61/146,292, filed Jan. 21, 2009, and U.S. Patent Application No. 61/183,571, filed Jun. 3, 2009, the contents of which are incorporated herein by references as part of the present disclosure.

### BACKGROUND OF THE INVENTION

1. Field of the Invention

The present invention relates to systems and methods for monitoring human performance. More particularly, the present invention relates to improved systems and methods for monitoring human performance in a variety of activities, such as swimming, wherein the systems and methods may operate in a variety of states by incorporating sensing equipment to capture performance information, and utilizing hardware and software to provide feedback and analysis.

2. Description of the Related Art

There are many solutions available today for automatically capturing information and data pertaining to certain types of human performance, such as running, cycling, and walking. Information such as route traveled, overall pace, cadence, average pace, peak pace, and other attributes may be captured and provided as feedback to the user. In some cases, a subset of the captured information is provided as feedback in real-time, while all of the information may generally be available for eventual download to a training log or analytics software package. By automatically capturing performance information, existing solutions can significantly reduce the burden on the user to manually capture performance data. In addition, performance monitors and training logs can serve as a source of motivation, by providing the user with the ability to track performance over time, assess areas for improvement, and set goals. Such solutions can utilize one or more of a variety of sensors, such as accelerometers, gyroscopes, magnetometers, altimeters, Global Positioning System (GPS) sensors or strain gages, to automatically capture information or data pertaining to performance.

Such systems are most commonly available for endurance athletes, such as runners and cyclists, to help them track and improve their performance. Examples of such systems include bike computers that display speed, distance, and cadence; GPS running loggers that track location, elevation, and pace; and bike power meters that track an athlete's power output (for example, in watts) over the course of a workout. In the modern, high-tech world of sport, these training aids can be indispensable.

Recently, other solutions have been developed as generalized performance monitors that measure overall activity. These are essentially sophisticated pedometer-like devices. Unlike existing solutions for running and cycling, these types of solutions do not capture specific performance metrics such as power generated, or route traveled.

While automatic performance monitoring solutions are growing in popularity and use, existing solutions lack the ability or capacity to monitor multiple specific attributes of human performance. Solutions available today either monitor a very specific activity, such as running or cycling, or a very generalized set of activities, such as movement. As a result, individuals who want to monitor different activities, such as running, cycling and walking, often need to use different performance monitors for each activity. In addition, available solutions generally lack the ability to set detailed performance goals that can be uploaded to a performance monitoring device and used to guide the user through a workout or activity session. Existing solutions that do support goal-setting features are typically limited to goals such as pace, total time of workout, or distance. Furthermore, these solutions are designed to monitor a certain set of performance characteristics from a given specific location. For example, running GPS devices require the user to wear a GPS sensor (generally on the arm or wrist) to automatically track the total distance run by the user, pace information, and the route taken by the user. However, many such devices cannot be attached to the leg of the runner, for example, to measure or monitor running efficiency.

One particular activity in which automated performance systems are lacking is swimming. Swimmers, like other endurance athletes, need to balance several critical components of their training regimen in order to improve fitness and, if desired, achieve optimal performance. However, unlike participants in nearly every other endurance sport, swimmers lack an automated solution for tracking and evaluating their performance, and are often burdened with the need to keep track of basic performance information such as counting strokes while focusing simultaneously on technique and execution throughout their workout, or are forced to use imprecise techniques such as using total time as a proxy for distance and average pace. Swimmers and coaches who choose to train and compete in an aquatic environment are frequently limited to rudimentary solutions such as the basic stopwatch. As a result, people who swim regularly for fitness and improved health, or train to compete, often do not have the same level of insight into their own performance and technique as do athletes in other sports. The development for more robust solutions for swimmers has been stymied by the inability to accurately measure and translate a swimmer's movement into accurate performance metrics, and also the inability to leverage wireless technology, such as GPS, in an aquatic environment.

Swimmers who strive to improve their performance understand that technique and form are critical components that impact their overall efficiency. For example, it has been shown that the performance of novice swimmers who swim the breaststroke tends to suffer from excessive drag, which may be caused by improper technique. While it is commonly understood that the point of greatest deceleration generally occurs during the leg tuck phase of the breaststroke, obtaining a direct measure of that deceleration is difficult with solutions available today.

Analyzing patterns of motion in athletes such as swimmers and measuring accelerations and velocity is a difficult challenge that often leads to imprecise measurements or incorrect conclusions. For swimmers, a common solution today for understanding technique and form, and their impact on performance, is through the use of underwater video cameras. While these solutions are effective in supporting observation of technique and form under the water, they are generally expensive and difficult to set up, can require manual interpretation of the captured performance data, and may not capture key aspects of the performance related to technique and form such as the acceleration of the swimmer's arms, rotation of the torso, and other metrics. A solution that can automatically capture fundamental performance information of an athlete (such as distance swum, pace, stroke count, and distance per stroke of a swimmer), while providing insight into the athlete's technique and form, would be valued by all types of athletes, including fitness-oriented individuals who strive to maintain a healthy living style, as well as elite, competitive athletes who measure performance increases in tenths of a second.

With motion sensor technologies such as accelerometers, gyroscopes, digital compasses, and magnetometers becoming more robust, smaller, and cheaper, there is an opportunity to directly measure the motion of an athlete, such as a swimmer, and to capture and derive critical performance information. For example, a standard microelectromechanical system (MEMS)-based accelerometer, such as the LIS302DL, can be used to track the movement of an athlete, such as a swimmer's arm across many swimming styles or strokes. Using standard signal processing techniques combined with heuristics and stochastic methods to interrogate the characteristics of the signal, it is possible to isolate and analyze key components of a swimmer's movement.

The present invention is directed to a performance monitoring system that uses a primary device and, optionally, one or more secondary devices, to automatically capture and track performance information in a variety of states, and is versatile enough to be utilized to monitor a variety of locations on a user's body and objects within the user's environment during the performance of a variety of activities. The present invention can also provide insight into the technique and form of an athlete by analyzing and identifying acceleration patterns and timings of phases within a multitude of events, such as each of the four major stroke types (butterfly, front crawl, back stroke, and breast stroke) or other strokes of a swimmer, and can also accept a plurality of parameters that are used to configure the primary device and other sensing devices to generate appropriate performance data. Examples of such parameters can include biometric data such as the weight or height of the user (to calculate calories burned over a given performance session), the size or length of the pool, track or route (to calculate the total distance traveled), or any other preferences that may impact the user interface and representation of data on the device (such a measurement unit preference, i.e., metric or imperial).

The market and opportunity for sophisticated systems and methods for automated performance tracking is readily apparent for all human activities in general, and for swimming in particular. Athletes who train at every level could benefit from a multi-state performance monitoring system and method that can capture performance data and provide both real-time feedback and long-term tracking and analysis.

### SUMMARY OF THE INVENTION

The present invention is directed to a multi-state performance monitoring system for capturing specific performance information or data from a variety of locations on a body and objects within the user's environment while relating to a variety of activities and providing real-time feedback. In addition, the present invention can provide the ability to download captured data to an online training log or analytic system while supporting the definition and uploading of detailed workout goals. The present invention's unique functionality is made possible by real-time interpretation of “raw” sensor data, preferably obtained from three-axis accelerometers, which is first filtered and pre-processed to remove noise and identify relevant portions of the data stream. Then, the pre-processed stream is passed through a hierarchical system of pattern recognizers that ultimately discern large scale features such as strokes, turns and laps, beginning with the smallest recognizable (and interesting) units of motion. Such large-scale elements compose the basis of the present invention's model, and are aggregated in different ways for both real-time and retrospective viewing by the user. The present invention's unique functionality is also made possible by the ability to be placed into different states or modes, thereby capturing performance metrics in a wide variety of activities through a single device.

The systems and methods of the present invention utilize standard sensors to monitor human performance in a variety of activities, such as swim training, and provide detailed feedback that a user, such as an athlete or coach, can use to improve performance. The systems and methods are fully automated and provide real-time information to the user, and do not require any additional device or apparatus to capture data, other than those that are worn or carried by the user.

The systems and methods rely on detailed information obtained from body-worn sensors, such as accelerometers, to monitor human performance. Components of the multi-state performance monitoring system of the present invention may be utilized in a variety of states, e.g., placed in different locations on a body and used to monitor performance in one or more activities. Different states of the invention are enabled through the device by allowing the user to download or install different performance recognition modules that may be specific to the activity being monitored. Once a performance monitoring state is selected either manually or automatically, by inferring the specific type of activity, the information collected by the sensors is run through a series of software filters and algorithms to provide the user with a detailed analysis of his or her workout. In the particular example of swimming, the present invention permits a swimmer or a coach to monitor and/or evaluate an individual's swim stroke.

The systems and methods may include at least three different versions or operating modes. A “basic” version of the systems and methods of the present invention may provide basic functionality for monitoring human performance in an activity, while a “plus” version may provide basic functionality plus the ability to upload and track performance information over time. A “professional” version may provide the functionality of the “basic” and “plus” versions, along with the ability to analyze an individual's techniques, mechanics or efficiency, and also to compare the individual's performance results with those of others. Specifically regarding the activity of swimming, the “basic” version could count strokes and/or laps, measure distance traveled, speed, calories burned and/or time, while the “plus” version could permit a swimmer to track this information over time. The “professional” version could further provide detailed analytics on the swimmer's biomechanics, including swimming technique and form, perform stroke and drag analysis, calculate an overall efficiency score, provide recommendations for improving efficiency based on one or more sets of heuristics, and compare an individual swimmer's performance against his or her previous performances, or against those of other swimmers in a community. The systems and methods of the present invention can thus capture detailed performance data to allow a user to identify critical areas for improvement, and assess and/or diagnose patterns that may be limiting the individual's performance. The systems and methods may also be integrated with other monitoring systems and methods, such as heart-rate monitors.

The present invention can include a performance monitoring system comprising a primary sensing device, and, optionally, one or more secondary sensing devices for monitoring human performance. Aspects of the present invention may be integrated with an online tracking log; placed into multiple states for capturing performance data; enabled to generate certain performance events, through the use of a combination of heuristics and stochastic data analysis techniques; worn on different parts of the body to capture different performance data; utilized with gesture-based recognition techniques to invoke certain features (such as starting and stopping a chronograph) and to place the system into different monitoring modes.

Aspects of the present invention may also accept a plurality of parameters to configure the one or more devices for the activity or performance that is being monitored; capture motion and environmental information through a variety of sensors; attach to various strapping mechanism to fit different parts of the body; integrate with an online training log which can record data and information, and provide long-term storage and analytics on the data and information captured by one or more sensing devices; and also provide the ability to configure and set certain performance monitoring preferences on sensing devices. Aspects of the present invention may further be updated with new performance recognition modules.

### DESCRIPTION OF THE SEVERAL VIEWS OF THE INVENTION

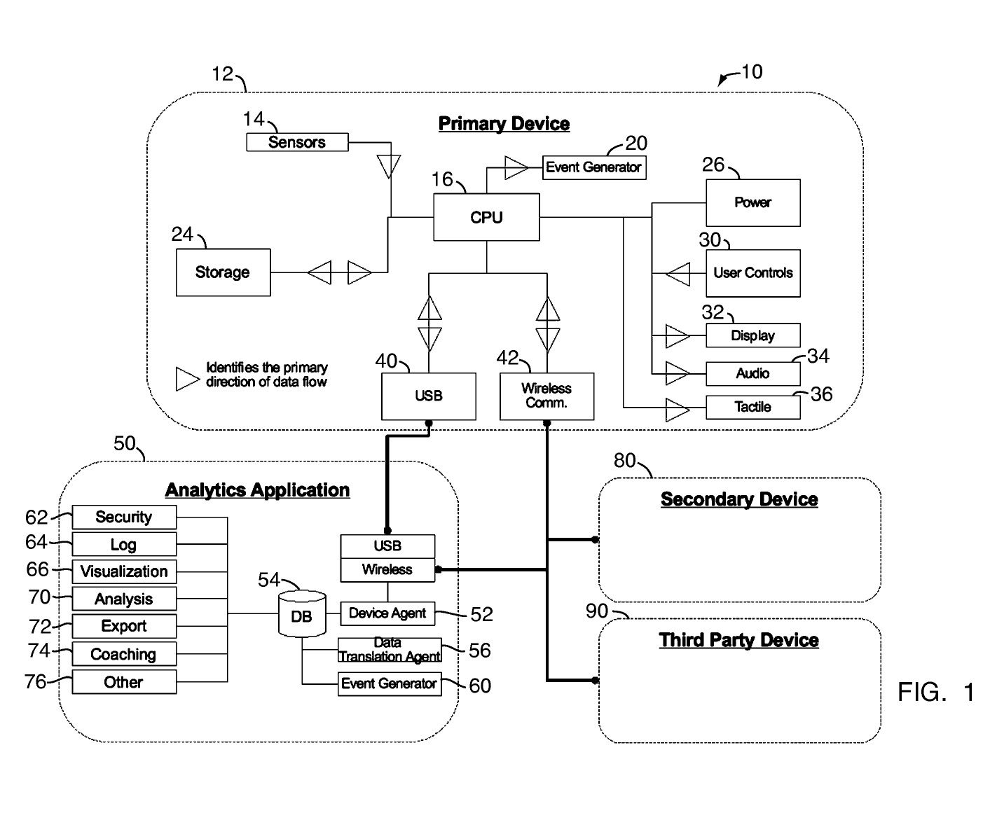

FIG. 1 is a diagram of hardware and software components of a multi-state performance monitoring system according to an embodiment of the present invention.

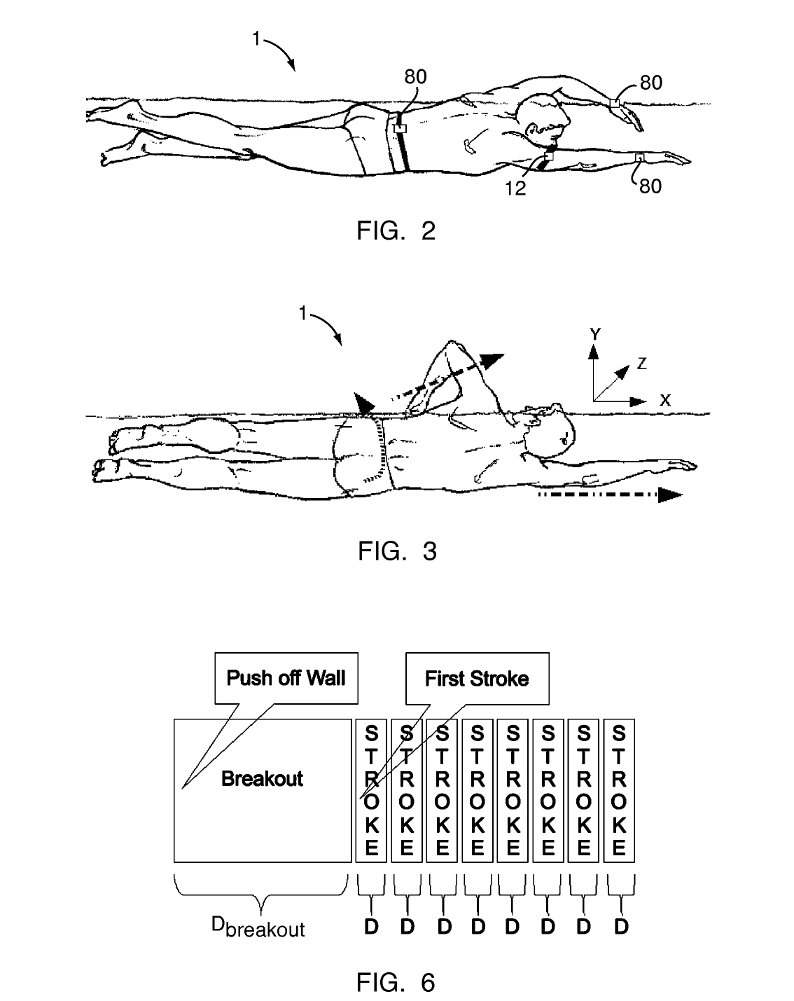

FIG. 2 is a diagram of an arrangement of components of a multi-state performance monitoring system according to an embodiment of the present invention, as arranged on a swimmer.

FIG. 3 is a diagram of the arrangement of components on the swimmer of FIG. 2, wherein the swimmer is engaged in a freestyle stroke.

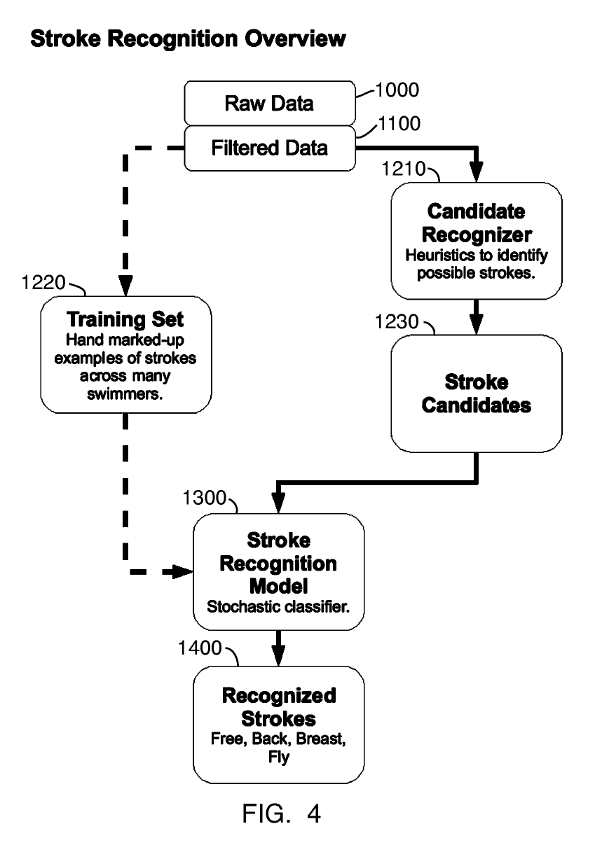

FIG. 4 is a flow chart for the recognition of a particular event of interest by a multi-state performance monitoring system according to an embodiment of the present invention.

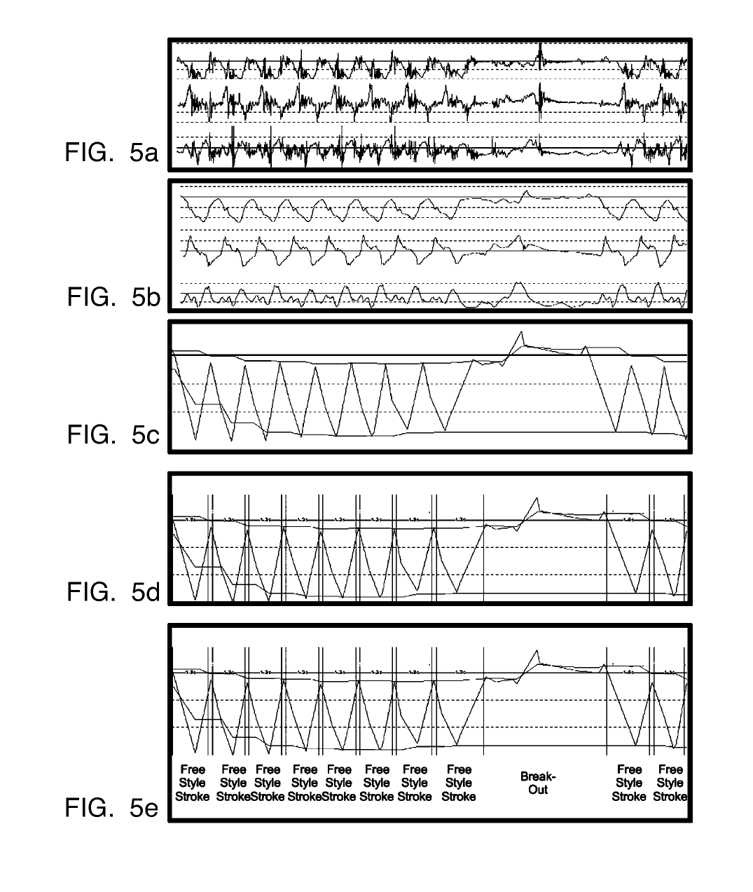

FIGS. 5a through 5e are a series of plots of data obtained by sensors and analyzed in a multi-state performance monitoring system according to an embodiment of the present invention, in various stages of processing.

FIG. 6 is a diagram of the various stages of a particular category of activity, as determined by a multi-state performance monitoring system according to an embodiment of the present invention.

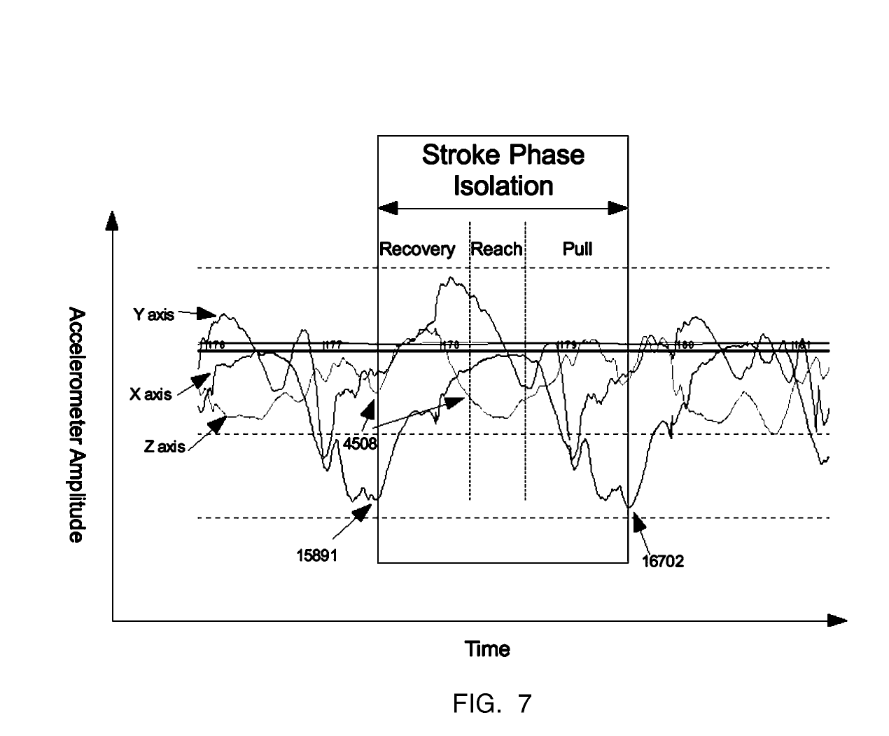

FIG. 7 is a series of plots of sensor activity for an isolated aspect of a particular category of activity, as determined by a multi-state performance monitoring system according to an embodiment of the present invention.

### DETAILED DESCRIPTION OF THE INVENTION

The present invention relates to improved systems and methods for sensing, monitoring, tracking and/or analyzing human performance in various states. As will be readily appreciated by those skilled in the pertinent art, the systems and methods disclosed herein are described in athletic applications, particularly in swimming applications, but may also be utilized or employed in many applications relating to human performance. The advantages and other features of the systems and methods disclosed herein will become more readily apparent from the following detailed description of the invention taken in conjunction with the drawings, which set forth certain currently preferred representative embodiments of the present disclosure.

Referring to FIG. 1, a multi-state system 10 for monitoring human performance includes a primary device 12, an analytics application 50, a secondary device 80 and a third-party device 90. The primary device 12 serves as a sensing device, data aggregator and communications hub for the system 10, and includes at least one sensor 14, a central processing unit (CPU) 16, an event generator 20, a storage unit 24, and a power source 26. The primary device 12 further includes user controls 30 and means for providing feedback to a user, including a display implement 32, an audio implement 34, and a tactile implement 36. The primary device 12 also includes one or more communications interfaces, such as a Universal Serial Bus (USB) interface 40 or a wireless communication interface 42, and may be sufficiently versatile to be placed on a variety of locations on a body to record information and data pertaining to the body's performance in a variety of activities.

As is set forth above, the primary device 12 shown in FIG. 1 features one or more sensors 14, which may capture information about not only the performance of an individual during an activity, but also the environment in which the activity takes place. The preferred type of sensor 14 to be utilized in the present invention is a three-axis accelerometer, which may capture movement characteristics such as velocity and acceleration with respect to three axes. Other sensors, such as one-axis or two-axis accelerometers, gyroscopes, magnetometers, GPS devices, digital compasses, force sensors or pressure sensors may also be used to improve the accuracy of the captured performance data, to provide additional information about the individual's performance, or to correct errors that may be resident in the processing of other sensors. Sensors may also be used to measure environmental conditions, such as temperature, humidity, and light intensity. A barometer or altimeter, for example, may be used to measure the altitude at which the individual performs some or all of the activity, for example, in order to adjust algorithms that measure intensity and calories burned, or for any other reason.

The CPU 16 is the central command unit onboard the primary device 12 shown in FIG. 1. The CPU 16 executes software algorithms and other code for capturing, analyzing and transmitting sensed data and other information, and provides control logic for the primary device 12, user controls 30 and feedback mechanisms 32, 34, 36. Additionally, the CPU 16 may also contain code for pairing one or more secondary devices 80 and/or third-party devices 90 with the primary device 12, or for sampling the various sensors 14 (e.g., accelerometers, magnetometers, gyroscopes) at a given sample rate. One example of a CPU 16 that may be utilized in an embodiment of the present invention is a 32-bit ARM-based microcontroller.

The event generator 20 is a software component that uses one or more techniques to translate “raw” data received from sensor 14 or other sensors into performance events, which can be either “atomic” (i.e., a discrete event that cannot be decomposed into other events) or “compound” (i.e., an event that is composed of one or more discrete events). The operation of the event generator 20 may also be driven based on the particular module or operating mode of the system 10, which may preferentially direct the event generator 20 to generate performance events chosen from a set or subset of particular events, based on the data obtained from the sensor 14. For example, when the system 10 is used by a swimmer, performance event data received from a sensor 14 (such as an accelerometer) could be translated into a single stroke event, or a series of stroke events, followed by a turn event or an end-swimming event. Depending on the configuration of the system 10 and the performance characteristics being monitored, i.e., the performance recognition module that is loaded and/or active, the event generator 20 may utilize a variety of approaches or techniques (such as a rules-based approach or a stochastic approach), or a combination of approaches or techniques, to process the data and translate it into one or more performance events. The event generator 20 is written in a programming language (such as ANSI C) so that it can be easily transferred to the analytics application 50, where it can perform a similar function.

The primary device 12 shown in FIG. 1 also includes storage unit 24, power source 26 and user controls 30. Data captured regarding the individual's performance is stored in storage unit 24, which can be a persistent storage unit such as Flash-type memory in MicroSD format, until it is deleted or overwritten. Power source 26 may be a rechargeable battery that provides power to the various components of the primary device 12. User controls 30 permit a user to start, stop or pause the capturing of data relating to workouts; to start or designate intervals of activity; to download or upload data; to view or review previously recorded performance data; or to configure the primary device 12 to the user's preferences. User controls 30 may be implemented in any combination of hardware and software.

The primary device 12 shown in FIG. 1 also features a variety of feedback mechanisms, including a display implement 32, an audio implement 34 and a tactile implement 36. The display implement 32 provides visual feedback to a user during configuration of the primary device 12 or system 10, uploading and downloading of data, initiating performance of and performing an activity, and after the activity has been completed. The audio implement 34 and/or tactile implement 36 may provide audible or tactile feedback or any other information. For example, during a workout, the primary device 10 may provide the user with a status report of the portions of a workout that have been completed, and what is left to be completed, or the user's progress in achieving one or more performance goals. For example, the primary device 12 may audibly instruct the user, “Interval 5 of 10 completed,” or “You are currently three seconds ahead of your target pace of 1:30 per 100 yards.”

The system 10 shown in FIG. 1 also features the ability to upload and download data and information between the primary device 12 and the analytics apparatus 50, and between the primary device 12 and one or more secondary devices 80 or third-party devices 90. As is shown in FIG. 1, the primary device 12 features a USB interface 40 and wireless interface 42, with the USB interface 40 providing the capability to upload and download data to and from the analytics apparatus 50, and the wireless interface 42 providing the capability to upload and download data to and from any secondary devices 80 or third-party devices 90. Those of skill in the art will recognize that other input/output (I/O) mechanisms and/or protocols may be utilized with the present invention, including a serial connection, Bluetooth, ANT+, infrared (IR), FireWire or others. In the system 10 shown in FIG. 1, a rechargeable power supply 26 may receive power from the analytics application via the USB interface 40, although other known devices or methods for charging power supply 26 may also be used.

As is shown in FIG. 1, the system 10 may optionally include one or more secondary devices 80, which may provide additional information regarding the performance, technique and form of the user during an activity. Secondary devices 80 may take the form of a primary device 12, and may contain the same or similar components as in the primary device 12, thereby allowing any single device to act as a primary device 12 or a secondary device 80, depending on its configuration. In the currently preferred embodiment of the present invention, if only a single device is used, it must operate in a “primary sensing” mode and be used as a primary device 12. Secondary devices 80 may be placed in different performance monitoring modes to capture specific performance measurements on different parts of the body such as the wrist, back and legs, and operate in conjunction with a primary device 12.

To reduce their size and/or complexity, secondary devices 80 need not contain certain components found in the primary device 12, such as a display implement 32, an audio implement 34 and/or a tactile implement 36, or the full complement of user controls 30 required to use the primary device 12. Additionally, in the currently preferred embodiment of the present invention, secondary devices 80 need not include a USB interface 40, as they may communicate with primary device via a wireless protocol, such as Bluetooth or ANT+, to transfer data to or from the primary device 12 during or following an activity. Similarly, the system 10 may optionally include one or more third party devices 90, such as a heart-rate monitor or a monitor of other physiological conditions, which may contain some or all of the components of a primary device 12 and operate in conjunction with the primary device 12 in a manner similar to that of a secondary device 80. FIG. 2 shows an arrangement of a primary device 12 and secondary devices 80 on a swimmer 1.

The analytics application 50 is a software application, such as an Internet-based and/or rich client application, that runs locally on a user's computer. The analytics application 50 provides data logging and analytics capabilities, and can feature several unique components to support users and/or coaches, or to link with other training logs, such as BeginnerTriathlete.com, Active.com, or TrainingPeaks.

The analytics application 50 may include a device agent 52, which is a software component that is executed on the user's local computer and serves as a communications and data bridge between the analytics application 50 and the primary device 12, as well as any secondary devices 80 or third-party devices 90. The main functions of the device agent 52 include verification, authentication, data transfer and configuration.

Specifically, the device agent 52 verifies the device and data prior to uploading and/or downloading data between the devices and the analytics application, and authenticates devices by verifying that the online account holder matches the ownership or credentials associated with the device in question. The device agent 52 also authorizes data, software and configuration information to be transferred between the analytics application 50, the primary device 12, and any other sensing devices. Such data may include “raw” information captured by sensors 14 on the primary device 12 or other devices; performance events generated by the event generator 20; activity plans that may be defined in the analytics application 50 and used to guide a user through an activity; or any other pertinent information, such as software or firmware updates, which may include new performance recognition modules and algorithms or enhancements to existing performance recognition modules and algorithms. The device agent 52 may also allow for configuration of the device preferences, including date, time, activity type, display settings, language preferences, and others. For the particular activity of swimming, the device agent 52 may permit a user to designate measurement type (i.e., yards or meters), pool size, weight, height, and other preferences that may be unique to swimming.

The analytics application 50 may also include a database 54 for storing a user's performance data, activities, preferences or other information. Data and information stored in the database 54 may be distributed to users or centralized, and may be relational, object-oriented or file-based. As is shown in FIG. 1, the analytics application 50 further includes a data translation agent 56, which may translate data from formats used by the analytics application 50 into other formats, such as Comma Separated Values (CSV), or those understood by other training applications, such as Active.com, BeginnerTriathlete.com, or TrainingPeaks.

The analytics application 50 may also include an event generator 60, which has the same abilities as the event generator 20 described above with respect to the primary device 12. The event generator 60 has the ability to reprocess “raw” data received from sensors 14 on the primary device 12, or on any secondary devices 80 or third-party devices 90, thereby allowing a user to take advantage of the generally superior processing capacities (e.g., CPU speed or memory) of a user's local computer or centralized server, as compared to those of smaller devices such as the primary device 12.

Once the data, information and/or processing events have been either uploaded from the primary device 12 or other devices, or created by the event generator 60, a variety of functions and components may be utilized by a user of the analytics application 50, including a security function 62, a log function 64, a visualization function 66, an analysis function 70, an export function 72, a coaching function 74, and any other functions 76 deemed necessary or desirable.

A security function 62 restricts access to the analytics application 50 and ensures the data stored therein will be protected by robust authentication and authorization security mechanisms, such as a unique pairing of a user's identification and password. The user may authorize others, such as a coach or training partner, access to his or her data through the security function 62.

Next, a log function 64 may provide a calendar-based view of the data and activities, and allow a user to navigate by year, month, week, and day to view planned and executed activities. In the particular example of swimming, the log may contain any of the following data elements for each workout: total time, average pace, total time per interval, pace per interval, total stroke count per workout, total stroke count per length, total lap count, total lap count per interval, total calories burned, or a comparison of planned workouts to actual workouts.

The visualization function 66 may provide a user with a two-dimensional (2D) or three-dimensional (3D) animated view of his or her performance, showing the key components of his or her form and technique. In the particular example of swimming, specific characteristics of the swimmer's technique and form are derived from the primary device 12, or optionally in combination with one or more secondary devices 80 or third-party devices 90. Based on pre-defined biomechanical information such as the swimmer's body type, length of arms, length of legs and length of torso combined with the data provided by sensors 14 and generated performance events, the visualization function 66 can show, for example, degree of hip rotation, time spent in a reach phase, elbow height during a catch and pull phase, position of the arm during recovery phase, position of the hip relative to the arms, and other key technique and form characteristics. In addition, the athlete will also have the ability to compare his or her animated technique against that of other athletes, such as those who demonstrate superior technique and form. Based on such a comparison, an automated set of recommendations may be provided to the athlete. Recommendations could be in the form of commonly understood swimming heuristics (such as rotating one's head with one's hips during the final moments of the pull phase) as well as suggested drills to improve technique (such as 3×100 yard repeats of a quasi-catch-up drill).

The analytics application 50 can also include an analysis function 70, which allows users to view their performance of an activity in a line graph format or Cartesian coordinate plane, such as where the X-axis represents time and the Y-axis represents one or more data elements from the log 64. For example, in the particular example of swimming, a swimmer may want to show stroke count in the Y-axis to see how his or her stroke rate changed over the length of the workout. The user can also select any subset range of the workout to view that chosen data element (i.e., stroke count) for the selected period of time. In addition there will several other analytic capabilities that may vary from product to product, and from activity to activity. Examples include a distance chart (i.e., a periodic chart showing the distance swam over a chosen time period, such as total yards swam over a week) or an intensity chart (i.e., a period chart showing the intensity levels of entire or parts of a workout over a chosen period of time, such as total intensity level per workout over a week). FIGS. 5a through 5e and FIG. 7 include examples of activity data displayed in a line graph format.

The analytics application 50 may also include an export function 72, which permits a user (such as an athlete) or coach to share his or her information (such as athlete profile, workouts, races, and the like) with other users or coaches.

The analytics application 50 may further include a coaching function 74, which permits individuals other than the user to define, distribute, analyze or manage a portfolio of users and their respective activities. In the particular example of swimming, the coaching function 74 may permit a coach to define workouts, such as from a set of pre-defined templates, or from a set of custom templates that are defined by the coach. Also, a coach may define workouts based on other existing workouts, thereby allowing mostly similar workouts to be created with out the burden of starting from an empty template. The coaching function 74 will also allow a coach to define workouts for a set of athletes that share common components (such as frequency) but differ in other components (such as intensity). The coaching function 74 will further allow a coach to assign a set of workouts to an athlete, including the date and time in which the workout should be executed, or to analyze the workouts of an individual workout, similar to the analytical features described above with respect to the analysis function 70. The coaching function also permits a coach to manage a group of athletes, including the ability to add athletes, remove athletes, rank athletes (e.g., based on performance), categorize athletes (e.g., based on variables such as coaching plan, type of athlete, or participating sport), or trade athletes with other coaches.

The components of system 10 may be utilized in a number of different states. For example, a triathlete may wear the same primary device 12 or, optionally, one or more secondary devices 80 and/or third-party devices 90 while performing in the swimming, cycling and running stages of a triathlon, and the devices may be used to capture aspects of the triathlete's performance in each of the stages. Additionally, the devices are also versatile enough to be worn on different parts of the body. For example, the same triathlete may elect to wear a primary device 12 on his or her wrist during the swimming stage, then on his or her bicep during the cycling stage, and finally on his or her ankle during the running stage, in order to capture performance information pertaining to each of these parts of the triathlete's body for each of these respective phases.

The components of system 10 described above, including primary device 12, analytics application 50 and/or any secondary devices 80 or third-party devices 90, may also be utilized in a variety of capacities for monitoring human performance. For example, in a “basic” version, an individual may choose to monitor only the most basic information, and may require the use of only a primary device 12 for this purpose. In a “plus” version, an individual may monitor his or her own performance using a primary device 12, and, optionally, one or more secondary devices 80 or third-party devices 90, and/or an analytics application 50 to upload and track performance information over time. In a “professional” version, an individual may require all of the functionality of the “basic” and “plus” versions, as well as extended features for analyzing form and technique, and for improving biomechanical efficiency.

For the particular activity of swimming, a “basic” system 10 may calculate basic workout information, including stroke count, distance, speed, calories burned or time. Embodiments of a “basic” system 10 may include an existing cellular telephone, portable computing platform or personal digital assistant (PDA), such as the iPhone, iPod Touch, BlackBerry Storm or Nokia 5500, which may contain sophisticated sensing capabilities and/or a robust computing platform for operating software, in the form of applications or other software, on such devices. Built-in sensors, expansion slots or other standard interface components may allow one or more other sensors 14 to be integrated with such mobile devices to create a robust sensing platform. Alternatively, a “basic” system 10 may include a primary device 12 in the form of a wristwatch-like device worn by a swimmer. Such a primary device 12 can include clock-based or stopwatch-like features, and, once activated (i.e., placed in “swim mode”) the device will be able to capture fundamental performance data as described above and display analysis results on the primary device 12, where the data and information would be saved until the next workout.

Also in the particular activity of swimming, a “plus” system 10 could utilize the same devices as in the “basic” system 10 described above, and include all of the functionality of the “basic” system 10 described above. A “plus” system 10 could also provide the ability to track workout information over time by uploading the information to an analytics application 50, including the historical reporting of individual workout information to enable a swimmer to improve over time.

A “professional” system 10 could have the capacity not only to monitor performance in real-time and over time, as in the “basic” and “plus” systems disclosed above, but also to provide detailed insight and analytics. For example, although many swimmers understand that proper technique is essential to improving performance, even with a conceptual understanding of the elements of a proper stroke, optimal body position, etc., it is nearly impossible for a swimmer to identify areas for improvement without the aid of a coach who can watch and assess a swimmer's form. A “professional” system 10 can close this gap by expanding upon the features of the “basic” and “plus” systems, including the primary device 12 and analytics application 50, to provide detailed insight and analytics on the swimmer's biomechanics including swimming technique and form. Similar to cycling power meters that exist in the market today (i.e., PowerTap, SRM, and Ergomo), a “professional” system can capture detailed performance data that allows a swimmer to identify critical areas for performance improvement. Specifically, a “professional” system can provide insight into form and technique (for example, during a freestyle stroke hip rotation, arm recovery position and speed, and duration of arm extension, relative position of both arms, position and speed of arm during pull phase and overall body position are critical components of a swimmer's technique and form), recommendations for improving bio-mechanical efficiency (for example, based on a set of pre-defined stroke-specific heuristics or through the development of a knowledge-base, the swimmer will be able to compare and assess her own form and technique to a higher standard), or a stroke efficiency score (for example, by automatically comparing the swimmer's technique and form against a standard, the analytics application 50 will be able to calculate a relative or absolute score that succinctly describes the swimmers efficiency).

Referring to FIG. 2 and FIG. 3, the following scenarios describe how sensor data (such as that received from a three-axis accelerometer) may be used to capture performance information from an athlete engaged in an activity. It is important to note that any specific references to acceleration patterns or degrees are provided for illustration purposes only. For these scenarios the following (starting) orientations of the accelerometers in relation to the device are assumed: X is the forward or backward direction, Y refers to the up or down direction, and Z refers to the side-to-side direction.

Referring to FIG. 2, a swimmer 1 is wearing a primary device 12 on his right bicep, and secondary devices 80 on his left wrist, his right wrist, and his torso. The swimmer 1 is engaged in the freestyle (or front crawl) stroke.

Referring to FIG. 3, when monitoring hip rotation during a freestyle stroke using a sensor mounted to the swimmer's torso, the swimmer's hips should be rotating about a central axis that extends to the swimmer's shoulders. Therefore, the optimal biomechanical measures to be observed during the freestyle stroke with a swimmer 1 using a system 10 configured as shown in FIG. 2 may be described as follows. In the X-direction, a negative acceleration should be observed, as there is no arm movement in an active catch or pull. In the Y-direction, acceleration due to gravity should be observed as the hips rotate, from 0 degrees (i.e., with the stomach oriented facing the floor) to a maximum of +/−45 degrees (i.e., with the stomach angled toward either side). Angular information may be derived or converted from the accelerometer or gyroscope data using conventional calculations. In the Z-direction, no dynamic acceleration should be observed, but a lessening of static acceleration due to gravity will be observed as the hips rotate.

However, in addition to optimal measures, some negative biomechanical measures may also be observed. Larger than expected declines in acceleration in the X-direction may indicate that the swimmer is losing too much momentum between arm strokes. Negative acceleration (i.e., hips moving towards the floor) in the X-direction, which commonly occurs when the swimmer lifts his or her head to breathe rather than rotating along the central axis, may indicate degraded performance due to significant drag. Finally, either positive or negative accelerations observed in the Z-direction can indicate that legs are “flaring” during the kick, resulting in additional drag and momentum in a non-forward direction.

Referring again to FIG. 3, when monitoring the left arm recovery phase during a freestyle stroke using a sensor mounted to the swimmer's left wrist, the left arm should be traveling up and forward with the forearm, wrist, and fingers in a relaxed position and the fingers just above the water line. At the beginning of the recovery, the primary direction of momentum of the left wrist is up, and, during the rest of the recovery and entry into the water, the momentum should be in a forward and slightly downward direction. Optimal biomechanical measures to be observed may be as follows. In the X-direction, an increase in positive acceleration should be observed during the first half of recovery, with peak positive acceleration occurring with the hand approaching water entry. In the Y-direction, an increase in positive acceleration should be observed, with a maximum angle achieved in the first third of the recovery. In the Z-direction, minimal acceleration should be observed. Some negative biomechanical measures which may be observed include, in the X-direction, a maximum angle achieved after the first third of the stroke, which may indicate that the arm is being lifted too high out of the water, resulting in inefficient form and delayed hand entry, and, in the Y-direction, significant acceleration (most likely away from the body), which may result in inefficient form and delayed hand entry.

Similarly, referring again to FIG. 3, when monitoring the right arm extension phase during a freestyle stroke using a sensor mounted to the swimmer's right wrist, the right arm moves into a fully extended reach position after entry into the water. When fully stretched, the right arm and hand serve as an “anchor” to support the rotation of the hips while supporting the catch and pull with the left arm. This is a key movement in the freestyle stroke, in that proper extension elongates the body and supports “front-quadrant” swimming technique while creating the critical potential energy stored in rotated hips (similar to a batter cocking his arms and shoulders and coiling away from the pitcher in baseball). Optimal biomechanical measures to be observed may be as follows. In the X-direction, a short burst of positive acceleration should occur while the right arm is entering the water and extending, followed by negative acceleration as the arm slows. Once the arm has reached maximal extension, little to no acceleration in the X-direction should be observed. In the Y-direction, a negative acceleration and nominal drop in angle (approximately 5 to 10 degrees) should be observed. In the Z-direction, no acceleration should be observed. Negative biomechanical measures may also be observed, including, in the X-direction, a lack of any measurable time in which there is no acceleration, which would indicate a lack of extension and glide. In the Y-direction, a large acceleration and angle change greater than 10 degrees would indicate that the arm is going too deep in the water, resulting in a sub-optimal condition in which the arm creates drag. In the Z-direction, any measurable acceleration indicates inefficient form and wasted energy, due to the arm's movement from side-to-side.

The following describes how an athlete may use an embodiment of the present invention to capture performance data. In this example, a primary device 12 will be worn on a swimmer's upper arm, and will have means to accept user input during a workout, and a display to show relevant information in real-time. However, it is important to note that actual placement of the primary device 12 on the body could vary. A decision to place the primary device 12 at different location such as the arm, waist, back, legs, etc., could be driven by and impact form factor design, the usability of the primary device 12, and the overall performance characteristics of the system 10. Different placement locations may also be associated with different states of the device, where each state implies one or more unique performance recognition modules and one or more sets of outputs specific to a location. A typical swim workout might proceed as follows.

A swimmer puts her iPhone containing a monitoring application on her bicep using an accompanying armband. The iPhone device includes internal sensors such as a three-axis accelerometer. To begin monitoring, the swimmer starts the application, and taps a “Start Workout” button on the user interface. At this point, the system begins recording and analyzing data on her motions. Next, the swimmer enters the pool and swims a few laps to warm up. The display shows the lap count, stroke count, total time, and total distance.

After stretching, the swimmer is ready to begin her true workout, which will consist of a set of six 200-yard intervals. At the beginning of each interval, she initiates another interval by tapping the “Start Interval” button. As she progresses through each interval, the interval count, lap count, stroke count, distance and time are displayed on the primary device 12. At the end of the workout, the swimmer taps an “End Workout” button on the user interface, and leaves the pool.

The swimmer then has the choice of reviewing summary-level statistics on the primary device 12 itself, or uploading the captured data to an analytics application 50. Data elements which may be reviewed can include, but would not be limited to, total distance swam, average speed, average stroke count per length, average speed per interval, average arm stroke count per interval, maximum speed per interval, etc.

The swimmer also has the opportunity to upload captured data from the primary device 12 to an analytics application 50 (either web-based or client-based). Data and statistics gathered from her workout are similar to the data that is directly available on the device but also includes detailed insight into the swimmer's form and technique. Using the logging and analytics software, the swimmer will also have the ability to compare her performance to past workouts and potentially other swimmers who use similar devices.

Events and interesting features of the individual's performance, such as strokes or stroke cycles of a swimmer, may be recognized using an algorithm and a sensor 14 (such as a three-axis accelerometer) in a primary device 12 positioned, for example, on an athlete's wrist. Sets of related features and their associated algorithms are combined into performance recognition modules associated with a state of the system.

While many types of sensors are useful and can be interpreted using similar techniques, the following example focuses on the use of an accelerometer. Generally, an accelerometer measures acceleration in a single direction, with the measured values being a combination of static acceleration (gravity), dynamic acceleration (movement) and some amount of error. Though it is often referred to as a single sensor, the three-axis accelerometer has three sensors positioned orthogonally so as to measure acceleration in the X-, Y- and Z-directions. Methods for discerning event types using accelerometers may include steps for identifying candidate events based on characteristics of accelerations, extracting a set of descriptive features from the candidate's waveform (for example, averages, minima or maxima), and using a statistical classifier such as a Hidden Markov Model (HMM), linear discriminants, neural networks or others, to classify and/or recognize an event. For swimmers, it is important to be able to distinguish between types of swim strokes, which can be recognized as periodic sharp accelerations either on a single axis, on multiple axes, or in the overall magnitude of the acceleration. Major stroke types include the front crawl (freestyle), backstroke, breaststroke and butterfly. Less common strokes (such as the sidestroke, the elementary back stroke, and others), as well as a number of drills that are “stroke-like,” that may also be considered for recognition.

In order to recognize patterns or events, the “raw” data sensed by one or more sensors 14 is preprocessed using both traditional and novel filtering techniques. Examples of such techniques may include a low pass filter, which can be used to attenuate high frequency portions of the signal, effectively reducing noise; a high pass filter, which can be used to remove a low frequency or static signal such as gravity, leaving only the dynamic acceleration; and a directional equivalence filter, which can be used to combine successive samples that are identical within a threshold, reducing the size of the data and limiting the impact of scale differences across swimmers. Which filters are to be applied may depend on the individual situation, such as the activity performed or the analysis sought. For example, a low pass filter that removes noise may not be desirable when seeking the high-frequency acceleration spikes caused by a swimmer's inefficient arm slapping the water (causing a splash).

The recognition of interesting features in a data stream may be accomplished using a hierarchy of recognizers, in a manner analogous to a speech recognition system that processes a stream of audio. In such systems, the most basic units of speech, called phonemes, are recognized first. In some cases, a single segment of audio may yield multiple phoneme interpretations, each with an associated probability. In a second stage, the phonemes are combined into words. Ambiguity in phoneme recognition may be resolved when the phonemes are considered in the larger context of words. Further, words can be combined into phrases and sentences, providing another set of semantic information that may be useful for disambiguation.

For example, when the systems and methods of the present invention are used to recognize a swimmer's strokes, the “phonemes” may be actions such as a single freestyle stroke, a single butterfly stroke, etc. These actions act as building blocks that may be combined into “words,” such as pool lengths, which consist of a sequence of strokes ending with a turn or rest period. For more advanced functions, the building blocks may be more basic, and considered individual parts of a stroke, such as the reach and the pull of the freestyle crawl.

Recognizing the features of a data stream may be accomplished using methods, such as intuitive heuristics (or a rules-based approach) and machine learning (or stochastic approach), depending on the state and configuration of the system 10 and the performance characteristic to be monitored. The simplest approach, intuitive heuristics, is based on descriptive patterns created by an analyst. In simple terms, these patterns might be described such as, for example, “more than 1.2 g acceleration in the X-axis accompanied by a Y acceleration between 0.15 g and −0.15 g and a positive Z acceleration.” Programmatically, these patterns can be implemented using a simple declarative language similar to parsing grammar, and tested against a data set that has been manually annotated with features of interest.

Rules may be composed of one or more base rules that perform logical tests on characteristics of signals received from sensors, and any number of base rules can be combined using Boolean operators to create more complex rules. The primary characteristics of a signal that are evaluated include signal amplitude and elapsed time. Many other characteristics could also be evaluated, including rate of change, comparison to past events in the form of averages or variances, amplitudes relative to a moving maximum-minimum window, and others. Each rule can be bounded by an elapsed time parameter, and has an associated priority value that governs the order of the rule evaluation. An example of using a rule to identify distinctive signal pattern from a three-axis accelerometer may be:

Minimum Duration=0.1

Maximum Duration=3.0

Priority: 7

Rule Specification: {z=−0.6} within 0.1 to 1.0 s {y<−0.75, z>−0.2} for 0.1 to 2.0 s within 0.1 to 2.0 s {z=−0.6}

where the Minimum Duration and Maximum Duration set lower and upper bound time limits on the signal being analyzed, and the Priority is a parameter that governs the order in which rules are executed.

The Rule Specification describes, in an understandable format, a series of conditions on the values of the X, Y and Z acceleration components. For example, the Rule Specification set forth above may be read as, “The rule starts with Z acceleration equal to −0.6 g. Within 0.1 to 1.0 seconds, Y acceleration must be less than −0.75 g and Z acceleration greater than −0.2 g. This condition must persist for at least 0.1 second, and not longer than 2.0 seconds. Then, within 0.1 seconds to 2.0 seconds, Z acceleration must again equal −0.6 g.

Alternatively, stochastic processing may used in combination or in place of rules. For example, a rolling average of the time between strokes, referred to as the stroke gap, may be calculated and stored in memory. After a stroke is identified, the next stroke is expected during a time range that is based on the current stroke gap value. The duration range may be, for example, −10% of the stroke gap up to +10%. This type of stochastic processing can be combined with a rules-based approach to increase the confidence that a stroke has occurred and has been correctly recognized.

In other instances, a stochastic-only approach to recognize interesting events or features may be required (e.g., to improve performance of a sensing device and reduce power consumption). An example of a stochastic-only approach could include the use of classification algorithms (e.g., the use of support vector machines, HMMs, neural networks or linear discriminant models) to identify certain types of swimming strokes based on combinations and values of signal characteristics bounded by a time interval. For example, within a pre-determined or calculated time interval, the following signal characteristics could be extracted: maximum amplitude, minimum amplitude, average amplitude, average amplitude change, a series of amplitude changes, etc. In addition, these signal characteristics could be combined with other elements, such as the configuration parameters from the device or information received from other sensors.

These classification algorithms (“classifiers”) are generally created, at least in part, via supervised machine learning. Machine learning techniques, specifically for classifiers, work by training a statistical model on a set of correct examples, and then using the resulting model to evaluate new examples. Each new example (or “observation”) exposed to the model is given a probability that it matches a “class,” or a grouping of similar examples, called the “training set.” One type of classifier that has been demonstrated in the motion recognition domain is the HMM, which is also used extensively in speech recognition. In the case of the HMM, as is true with many classification techniques, the observation converted to discrete feature vectors containing descriptive elements of the underlying data.

An example of using a performance recognition module to recognize swimming strokes using an embodiment of the present invention is described below and shown in FIG. 4 and FIGS. 5a through 5e. In this example, at step 1000 in FIG. 4, “raw” data is obtained from a sensor 14, such as a 16-bit three-axis accelerometer, sampled at a frequency of 30 Hz. Next, at step 1100, the “raw” data is filtered using a low-pass filter with a cutoff of 1 Hz. At steps 1210 and 1230, heuristics are used to identify “candidate strokes,” i.e., portions of the filtered signal that may represent a stroke. One method for recognizing “candidate strokes” (for example, for a front stroke) includes the following two steps. First, on the axis parallel to the long axis of an athlete's arm, remove all data points that are not either a local maximum or a local minimum, over 45 samples. For a 30 Hz sampling frequency, this corresponds to a 1.5 second period. Second, identify as a candidate any sequence of maximum-minimum-maximum with a total acceleration change of, for example, at least 1 g, and a duration of less than three seconds. Then, at step 1300, a previously trained statistical model may be used to evaluate the candidate. The model may be a simple set of linear discriminants or a more sophisticated model, such as an HMM or a neural network. Finally, at step 1400, the candidates are tagged with a stroke type (for example, freestyle, backstroke, breast stroke or butterfly).

Also as is shown in FIG. 4, in step 1220, classification algorithms such as HMMs, neural networks, linear discriminant models and others may be created using a “training set,” which consists of a set of observations or characteristics that are known to be members of a certain class. The classes could be defined broadly in an either/or format, i.e., “stroke” or “not a stroke,” or as members of smaller classes, i.e., “front crawl stroke,” “backstroke,” or others. Using the “training set,” as shown in step 1220, a model may be created based on the sample, known observations, and the model will attempt to classify new, subsequent observations based on what was learned from the “training set.”

FIGS. 5a through 5e are data plots reflecting the use of an algorithm, such as a performance recognition module, to recognize strokes, and show examples of data obtained by following the process described above and in FIG. 4. In FIG. 5a, “raw” data obtained directly from a three-axis accelerometer is plotted for a swimmer who is wearing a sensor 14 on his or her right wrist and swimming the front crawl stroke. Next, in FIG. 5b, the “raw” data is shown after low pass filtering with a 1 Hz cutoff frequency. This simple technique removes high-frequency elements and generally smoothes the signal. In FIG. 5c, the filtered data is shown as stripped down to only its extreme values, minima and maxima. Stripping the data to its extreme values is another example of filtering, and is accomplished via a moving window within which the maximum and minimum data values on an axis are identified. The values shown in FIG. 5c are plotted for the X-axis only. In FIG. 5d, the data is shown with regions of the data identified as “candidate strokes.” Using heuristics, “candidate strokes” are identified, for example, as any sequence of “max-min-max,” or a maximum followed by a minimum followed by a maximum, wherein the total change in acceleration is greater than a predetermined threshold. In FIG. 5e, a recognition model is applied to the “candidate strokes” to classify them as a known stroke type (based on training data or other information) or as “not a stroke.” The results are candidate regions that are tagged as a stroke type, i.e., “recognized strokes,” as in step 1400, described above.

Traditionally, sports watches require the wearer to press a button to mark the beginning and the end of an interval. Pressing a button carries the advantages of accuracy (to the extent that the athlete is accurate) and predictability (the user marks an interval when expected). For some athletes, however, who are not accustomed to marking intervals and whose workouts may include many intervals, marking intervals manually may be disruptive to their workout.

The components of system 10 may be used to infer the beginning, conclusion and other aspects of events or performance intervals based on sensed information or data regarding the actual performance itself. For example, in swimming, the start of an event can be identified through a push-off from a wall, a gap in stroke count, a change in magnetic direction, or other strategies or combinations of strategies. To calculate the start of a lap from a push-off from a wall, a signal appears as a sharp acceleration in the axis parallel to the long axis of the swimmer's arm. This assumes that the swimmer's arms are extended in front of him or her, as is common. During this acceleration period and immediately thereafter, the sum of acceleration in the other two axes should be approximately 1 g, or gravity. Once this pattern is identified, the end of the prior length can be delineated as the start of the acceleration (where the swimmer's feet are in contact with the wall). In order for an algorithm to actually recognize a push-off pattern, a combination of rules-based (heuristic) and stochastic methods may be applied. Simple rules can identify candidates, and may be confined to operate within gaps in strokes. Once a push-off candidate is identified, statistical tests can accept or reject the candidate.

In the particular example of swimming, gaps in strokes may be used to identify turns at the ends of a lane, where a push-off from the wall is not directly discernible. In this case, a gap between strokes that is greater than some factor of the average stroke gap (e.g., the factor could be three) will be counted as a turn. Since the exact instant at which the wall is touched may not be known, an average factor, calculated from observations of many swimmers, can be used to divide the gap between the previous length swum and the length that is just starting. Using a two-axis or three-axis magnetometer, turns at the ends of lanes can also be detected. In using a magnetometer to identify the start of a lap, the largest challenge is the circular motion associated with long-axis strokes (back and front crawl). Here, the magnetometer needs to be sampled at known positions of the arm (e.g., extended forward). When using magnetic direction, the exact delineation between lengths is difficult to detect. However using heuristics based on observation of many different swimmers, an approximation can be made. This type of turn detection is particularly useful for non-stroking drills (such as kick drills), where stroke gaps are not present.

The present invention may also infer the beginning and end of an interval based on the recognition of certain events, such as strokes or laps. Most swim workouts are divided into “sets,” or a contiguous sequence of lengths separated by a short period of rest. For example, a workout may begin with 500 m of warm-up, followed by 6×200 m at a faster pace. In this example, the 500 m and each of the 200 m repeats are sets. Many competitive swimmers swim 3000 m or more in a workout, and hence many sets. Note that the terms “interval” or “intervals” are often used in other endurance-based sports such as running and cycling, and are conceptually equivalent to the terms “sets.” Given that some swimmers also run and cycle (e.g., triathletes), the term “sets” and “intervals” may be used interchangeably here.

The present invention may infer intervals in a number of ways. For example, using a rest time threshold method, the duration of periods of “not swimming” would be calculated and compared to a user-specified threshold (e.g., 5 seconds). Any rest period over the threshold would be considered an interval boundary. Key considerations for this method include the ability to discern rest periods or periods of non-swimming, or the ability to distinguish a breakout period from rest, given a user-specified rest period that may be shorter than the total time between a swimmer's last stroke of one length and the first stroke of the next length, which may include a long period of swimming without strokes due to the push-off. Gestures, such as tapping a pool wall twice, could be recognized by sensors 14 in a device, and used to mark interval boundaries. Note that methods may be combined to increase the accuracy of the system inferring intervals. Also, note that these methods need not replace the option to press a button on the primary device 12 to manually mark an interval.

The present invention has the capacity to capture various sensor data, including acceleration signals across three axes, and provide insight into the performance of individual event phases across an event cycle. Acceleration data may be analyzed manually or automatically, depending on the type and level of analysis desired. To measure, for example, the total stroke cycle time in freestyle swimming, for example, an algorithm can be implemented in software to iteratively search for the greatest (negative) acceleration on the X-axis, which is parallel or in the direction to the swimmer's arm. Referring to FIG. 7, such a value represents the point at which the swimmer's arm is in the closest position to being aligned with gravity and, therefore, the end portion of the pull phase. The algorithm will then search forward in time and find the next point of greatest negative acceleration. The elapsed time between these points represents the total time it takes for the arm to complete one stroke cycle. The algorithm will iteratively calculate the stroke cycle times until an end of length or turn event is found. Note that other points from the same or different axis may be chosen to calculate the total stroke cycle time.

An algorithm may also be developed to isolate an event phase from a series of events. Referring again to FIG. 7, in another example of a freestyle stroke, the algorithm will again find the point of greatest negative acceleration along the X-axis. At the same time, the algorithm will find the nearest greatest negative point of acceleration (between a falling or negative acceleration and a rising or positive acceleration) on the Z-axis. This point represents the start of the recovery phase. The algorithm will then search forward in time along the Z-axis until it finds the next point that has an acceleration value equal to original value found in the Z-axis, which is the point at which the swimmer's arm is leaving the recovery phase and entering the reach phase. The total elapsed time between the two points on the Z-axis represent the time taken to start and complete the recovery phase.

Similar manual analysis and algorithms may be used to identify and isolate other aspects of an event. As a result, detailed analysis within and across athletes may provide insight into how an individual athlete can improve his or her performance. For example, a coach can calculate timings for each phase of a stroke from an elite swimmer, and compare the calculated timings to those of a novice swimmer. The comparison could reveal to the novice swimmer the parts or phases of a stroke that require improvement in order to more closely align with those of the elite swimmer. Further, over time, sets of standards or benchmarks may be established for different types of swimmers (for example, by age, skill level, body type, gender or flexibility), after which swimmers with similar characteristics should model their strokes. Additionally, similar analysis may be used to show how an individual swimmer's timing of the stroke phases changes. Negative changes in timing within a workout, for example, may be due to fatigue. Conversely, the swimmer may improve his/her fitness level and technique over time, showing an improvement in the timings of the stroke phases.

Various acceleration characteristics may also be manually or automatically calculated to provide comparison points within and across athletes.

One calculation could find the peak (positive or negative) accelerations within a phase of the event. For example, a swimmer with a greater acceleration peak in the X-axis during the pull phase off a stroke may have a more effective pull compared to a swimmer with a lower acceleration value. Another calculation may show the total change in acceleration within or across event phases. For example, a swimmer with a larger total acceleration in the Y-axis may indicate the swimmer has too much side-to-side motion during the stroke, which may result in movement that is not adding to, and may be taking away from, the athlete's performance goals.

The present invention may be utilized to calculate a variety of factors and numerical data for tracking and analyzing an individual's performance. For example, system 10 may include a performance recognition module that can be used to calculate an “efficiency score” by performing a weighted average calculation against possible negative (or positive) biomechanical measures, and the exact attributes to be included in the calculation, as well as the weights, are configurable and can be set by the user as a configuration parameter. To calculate a total efficiency score TES based on a number of negative biomechanical measures nbm, the measures may be divided into a number of predetermined ranges, with each range assigned a graduated numerical weight w to each of the ranges, and the actual value of the negative measure n may be identified. The actual value of the negative measure may be matched to the appropriate range by multiplying the actual value by the range weight, i.e., nᵢ·wⱼ. Optionally, a lower bound bm_floor and/or an upper bound bm_ceiling may be established for the range, such that a biomechanical measure could not fall below the lower bound or exceed the upper bound, in order to constrain or simplify the equation or the derived scores.

The total efficiency score TES equals the sum of the calculated values from each negative measure:

$$TES = {\sum }_{i = 1}^{nbm} {n}_{i} \cdot {w}_{i} = {n}_{1} \cdot {w}_{1} + … + {n}_{nbm} \cdot {w}_{nbm}$$

The total efficiency weight TEW is calculated by summing the weights of the selected ranges:

$$TEW = {\sum }_{i = 1}^{nbm} {w}_{1} + {w}_{2} + … + {w}_{nbm}$$

The efficiency score ES is then calculated by dividing the total efficiency score TES by the total efficiency weight TEW, or:

$$ES = \frac{TES}{TEW}$$

According to a method for determining a swimmer's stroke efficiency score ES in accordance with the present invention, a swimmer 1 may wear a primary device 12 and, optionally, secondary devices 80 on different areas of the body, for example, as is shown in FIG. 2. The placement of the devices on each area of the body may be selected to correspond with the technique attribute to be measured for efficiency. Next, each device's internal clock may be synchronized to the same time by the primary device 12, and placed in the appropriate performance-monitoring mode to capture the desired performance attribute, and then placed in an active monitoring state. The swimmer 1 will swim at least one length of a pool, for a minimum of 25 yards, and each sensor will then be placed in a non-active monitoring state.

The swimmer 1 or a coach may then log into an analytics application 50 and upload the data from the primary device 12 and any secondary devices 80 to the analytics application 50. The user of the system may select an efficiency scoring function, which synchronizes the data from each device according to time. The efficiency scoring function calculates a stroke efficiency score ES, for example, in accordance with the weighted average calculation described above, and displays the stroke efficiency score ES to the user.

An “intensity factor score” is a relative intensity score for an activity, for a subset of an activity, or across multiple activities. For example, an intensity factor score I_{score} may be calculated based on the following formula:

$${I}_{score} = \frac{V}{ES}$$

where I_{score} is the intensity factor score (i.e., a measure of the athlete's effort level), V is the distance traveled per some unit time (i.e., 100 yards/1:25), and ES is an efficiency score, as described above. Based on the formula set forth above, I_{score} will increase with speed and decrease with greater technique efficiency.

An intensity factor score is largely useful for an athlete to compare his or her intensity across workouts or segments of a workout. However, with the addition of a constant that describes the individual's physical characteristics such as weight, height, body mass index (BMI), etc., the formula can be modified to provide an effective comparison across individuals. In this form, such a “power score” is analogous to measures of power per unit mass (e.g., watts per kilogram) used in sports such as cycling, and may be calculated as follows:

P_{score}=I_{score}·B

where P_{score} is the power score, I_{score} is the intensity factor score, and B is the athlete's body constant, which describes the athlete's physical characteristics as they relate to the particular activity.

The information set forth above may be used by an individual, or by both an individual and a coach, to assess and diagnose behaviors and patterns that may be limiting an individual performance. Furthermore, the individual and/or the coach will be able to observe changes in performance over time as it relates to the individual's form and technique (e.g., in the particular example of swimming, by reviewing changes in drag and efficiency scores).

The components of system 10 may also be used to calculate aspects of events, such as swimmer's stroke count, which is defined at the number of strokes taken over a given distance. When swimming in a pool, stroke count is generally calculated as the total number of strokes taken to complete one length (swim from one end of the pool to the other).

In the event that a swimmer is wearing a single primary device 12 on either arm, then stroke cycle becomes the primary measurement, and stroke count is derived from stroke cycle, which is defined as the total number of times the stroking arm with the primary device 12 completes an entire stroke. For example, in freestyle or the front crawl, a stroke cycle is completed when an arm completes all four phases of the stroke: reach, catch, pull, and recovery. To derive the total stroke count from the stroke cycle for strokes such as the freestyle or front crawl, the stroke cycle is multiplied by two, because each stroke cycle in the freestyle stroke includes one stroke by the left arm and one stroke by the right arm. However, in certain situations, the calculated stroke count based on stroke cycle will be plus or minus one, depending on which arm starts stroking first and which arm ends with a stroke.

Examples of calculating a stroke count from stroke cycles in the freestyle or front crawl are demonstrated below. The following four conditions assume that the primary device 12 is placed on a swimmer's right wrist. In the first condition, assume that the swimmer's first and last strokes are performed with the right wrist. If the total number of stroke cycles is N=10, then the total derived stroke count is 2N=2(10)=20. The actual stroke count, however, is 2N−1=19, because the swimmer began and ended with strokes of the right wrist, which was wearing the primary device 12.

In the second condition, assume that the swimmer's first stroke is with the right wrist and the swimmer's last stroke is with the left wrist. If the total number of stroke cycles is N=10, then the total derived stroke count is 2N=2(10)=20. The actual stroke count is 2N=20, because the swimmer began with a stroke of the right wrist, which was wearing the primary device 12, and ended with a stroke of the left wrist. Similarly, in the third condition, assume that the swimmer's first stroke is with the left wrist and the last stroke is with the right wrist. If the total number of stroke cycles is N=10, then the total derived stroke count is 2N=2(10)=20, and the actual stroke count is 2N=20, because the swimmer began with a stroke of the left wrist, and ended with a stroke of the right wrist, which was wearing the primary device 12.

In the fourth condition, assume that the swimmer's first and last strokes are performed with the left wrist. If the total number of stroke cycles is N=10, then the total derived stroke count is 2N=2(10)=20, but the actual stroke count is 2N+1=21, because the swimmer began and ended with strokes of the left wrist, which was not wearing the primary device 12.

Note that for “short axis strokes” (i.e., breast stroke and butterfly), where both arms move in unison perpendicular to the long axis of the body, the stroke count is equal to the number of stroke cycles.

The components of system 10 may also be used, for example, to calculate a pace, which is defined as a unit of time over a unit of distance. In running, it is common to calculate one's pace in terms of minutes per mile. Likewise, swimmers often measure pace in terms of minutes or seconds per 100 yards or meters. When placed on the wrist, the primary device 12 will calculate pace based on the time it takes to complete a single length. The input parameters for pace calculation include a start-of-length performance event, an end-of-length performance event, and the length configuration parameter (e.g., a length of a track or a pool, which may be inputted by the user).

The following equation illustrates how pace may be calculated during a swimming session. Pace equals the time T required to complete a distance D, divided by the pace preference P (e.g., 100 yards) parameter set by the user, and divided by D, or (T/D)·P. Note that once a pace parameter is recorded for each pool length, an average pace can be calculated for each set or interval and the entire workout. Additionally, the total distance swum D may be calculated by multiplying the length of the pool L (provided by the swimmer) by the number of lengths C completed, or D=L·C. The total number of laps, where a lap is defined as two pool lengths, may be calculated as the number of completed lengths divided by two.

Distance per stroke (or stroke cycle) is a commonly used metric related to a swimmers technique. Generally, the larger the distance per stroke, the more efficient a swimmer's technique. It is seldom, however, that an actual distance per stroke is calculated, at least partly due to the fact that it is difficult to separate the “stroking portion” of a length swum from the distance covered during the push-off (i.e. the distance from the point of pushing off from the wall to the point in which the first stroke is taken, sometimes referred to as the point of breakout). A diagram of the push-off portion, or breakout, and the individual strokes swum by a swimmer is shown in FIG. 6.

Once the length of the push-off portion is known, the true distance per stroke can be accurately calculated as D=(L−D_{breakout})/C, where D_{breakout} is the breakout distance, C is the count of strokes, L is the pool length and D is the distance per stroke.

Using a wrist-mounted sensing and computation platform, the breakout distance D_{breakout} can be accurately estimated in several ways. For example, assuming that the time of the start of the length and the time of the first stroke are known, the breakout distance D_{breakout} can be estimated as follows:

D_{breakout}=(Tₛₜᵣₒₖₑ−Tₛₜₐᵣₜ)·A·F

where Tₛₜᵣₒₖₑ is the time of the first stroke, Tₛₜₐᵣₜ is the time of the start of the length, A is an average speed calculated over the previous length (or some other set of lengths), and F is a correction factor based on observations of many breakout phases across many swimmers.

Additionally, the breakout distance D_{breakout} may also be accurately estimated using integration, by identifying the start time of the push-off from the wall, identifying the start of the first stroke cycle, integrating the acceleration signal parallel to the direction of travel over time (assuming that the swimmer's arms are extended) to determine a velocity, and integrating the velocity again to yield a distance. This simple approach can be refined, as can many of the aspects of the present invention, using a magnetometer or gyroscope, and combining the two signals using a technique such as Kalman filtering.

When calculating the distance per stroke of a swimmer in open water, for example, the distance per stroke may be varied based on a multiplicative factor based on the swimmer's intensity, as compared to either a baseline (derived during calibration) or a global average. As intensity increases, the adjusted distance per stroke will increase, and as intensity decreases, so will distance per stroke. Intensity can be calculated as a function of stroke rate (i.e., strokes per minute), stroke power (which may be estimated using the acceleration of the arm), or a combination of the two, for example.

Further, the sample rate and accuracy of the underlying sensors and/or analog-to-digital conversion may impact the resulting calculation. (For example, a higher sampling rate may provide better calculation results.) In a preferred embodiment of the present invention, the system 10 can recognize a turn and then increase the sampling rate (for example, to 1000 Hz) for the estimated time to complete the breakout or until a first stroke is recognized. Such an approach would have the advantage of providing a high sample rate to improve the results of integrating the accelerometer signal as described above while reducing CPU, battery, and storage requirements for the other aspects of the swim.

The system 10 may also be used to calibrate the sensors. In a calibration state, a primary device 12 and/or a secondary device 80 will measure the number of stroke cycles over a known distance. By dividing the distance traveled by the stroke cycle count, an average distance-per-cycle is obtained. When placed in the calibration state, the swimmer's intensity (as a function of stroke rate or stroke power) will be measured. Steps for calibrating the system 10 may include placing the primary device 12 in calibration mode, beginning the calibration, swimming a known distance (e.g., 100 yards), stopping the calibration, and accepting or rejecting the obtained distance-per-cycle value. If the distance-per-cycle value is rejected, the process may be repeated.

In order to optimize their performance in a particular stroke, swimmers must develop proper technique. In front crawl, for example, coaches may provide feedback and assign swimming drills to develop and refine certain phases of the stroke. When a swimmer does not extend properly during the reach phase of a front crawl, a coach may assign various drills such as the “catch-up drill” in order to train the swimmer to extend more during the reach phase.

Those of skill in the art will recognize that the systems and methods disclosed herein may be used to calculate a pace variance, and also a stroke count variance, of an interval. To calculate a pace variance for an interval composed of a number of lengths, the system will store the time that was required to complete each length. At the end of an interval, the system will sum the squares of each length time and divide the total by the number of completed lengths, to determine the total pace variance of the interval. To calculate a stroke count variation for an interval composed of a number of lengths, for each length, the system will store the total stroke count. At the end of the interval, the system will sum the squares of each stroke count, and divide the total by the number of completed lengths, to determine the total stroke count variance of the interval.

The systems and methods may also be used to calculate the force produced by an athlete, which can provide valuable information about the intensity and strength of the part of the athlete's body that is being sensed or monitored. As is known to those of skill in the art, the formula for calculating Force is F=m·a, where F is the net force (for example, in units of Newtons, or N), m is the mass of an object (for example, in units of kilograms, or kg) and a is the acceleration of the mass (for example, in units of meters per second squared, or m/s²).

The present invention may analyze components of the acceleration signal received from a sensor (for example, an accelerometer) from one or more axes to calculate a “force score,” equal to the product of mass and acceleration. Several approaches may be undertaken to calculate a “force score” applicable to a workout, or a given portion of a workout. The algorithms described below may be used to calculate the force applied by a swimmer in the pull phase of a stroke, but other phases of a stroke, other parts of the body, and other activities may also be monitored to calculate such a “force score” (e.g., the torso or the ankle may be monitored to calculate the “force score” for torso rotation or kicking force of a swimmer, respectively).

The present invention may identify the major phases of an event through the identification of acceleration patterns (such as through the use of heuristics or machine learning patterns). By isolating, for example, the pull phase in the front crawl stroke, acceleration may be measured in at least the following two ways. First, the present invention may determine the absolute value of the total acceleration Aₜₒₜₐₗ during the pull phase in the X-axis (the axis going through the arm) of stroke T by subtracting the greatest positive acceleration value from the greatest negative acceleration value. Alternatively, the present invention may calculate the total acceleration Aₜₒₜₐₗ by determining the acceleration A_{X}, A_{Y} and A_{Z} on each of the three axes X, Y and Z (for example, by using a three-axis accelerometer), and calculating the magnitude of the total acceleration Aₜₒₜₐₗ according to the following equation:

Aₜₒₜₐₗ=\sqrt{A_{X}²+A_{Y}²+A_{Z}²}

Once the acceleration is calculated, a “force score” may be determined using an appropriate measure of the mass (for example, when the sensing device is mounted on a wrist and used to calculate total acceleration, the mass of an arm may be used) and for the drag caused by water on the surface of the arm, according to the equation F=m·Aₜₒₜₐₗ+F_{d}, where F_{d} is the force required to push against the drag of the water, and is defined by the “drag equation”:

F_{d}=½ρu²C_{D}A

where F_{d} is the force of drag, ρ is the mass density of the fluid, u is the velocity, A is the surface area, and C_{D} is the drag coefficient.

Masses and surface areas of limbs or other portions of the body may be estimated by any known methods, including those discussed in C. E. Clauser et al., Weight, Volume and Center of Mass of Segments of the Human Body, Aerospace Medical Research Laboratory, Aerospace Medical Division, Air Force Systems Command, Wright-Patterson AFB, August 1969. Alternatively, one may assume that the mass of the limb (such as an arm) remains constant across measurements (i.e., strokes, sets or workouts), and set the mass equal to 1. Such an assumption removes the complexity of needing to calculate the mass of the limb, but has the drawback of not being able to calculate and compare “force scores” between and among different athletes (such as swimmers).

Although the disclosure has been described herein using exemplary systems, techniques, algorithms, and/or processes for implementing the present disclosure, it should be understood by those skilled in the art that other systems, techniques, algorithms and processes or other combinations and sequences of the systems, techniques, algorithms and processes described herein may be used or performed that achieve the same function(s) and/or result(s) described herein and which are included within the scope of the present disclosure. Moreover, although the systems, techniques, algorithms and processes are described solely for use on athletes, they may be equally applicable for use on any particular humans or other animals, such as whales, racing horses or dogs, or animals in the wild.

Any process descriptions, steps, or blocks in flow diagrams described herein should be understood as potentially representing modules, segments, or portions of code which include one or more executable instructions for implementing specific logical functions or steps in the process, and alternate implementations are included within the scope of the preferred embodiments of the systems and methods described herein in which functions may be deleted or executed out of order from that shown or discussed, including substantially concurrently or in reverse order, depending on the functionality involved, as would be understood by those reasonably skilled in the art.

It should be understood that, unless otherwise explicitly or implicitly indicated herein, any of the features, characteristics, alternatives or modifications described regarding a particular embodiment herein may also be applied, used, or incorporated with any other embodiment described herein. Also, the drawings herein are not drawn to scale.

Conditional language, such as, among others, “can,” “could,” “might,” or “may,” unless specifically stated otherwise, or otherwise understood within the context as used, is generally intended to convey that certain embodiments could include, but do not require, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Although the invention has been described and illustrated with respect to exemplary embodiments thereof, the foregoing and various other additions and omissions may be made therein and thereto without departing from the spirit and scope of the present disclosure.

1. A multi-state system for monitoring performance of a body, the system comprising:
a first device mounted to a first portion of the body, the first device comprising a first sensor and a first computer processor,
wherein the system is adapted to be operated in a plurality of states;
wherein the first sensor is adapted to sense information pertaining to the performance of the body in at least one of a plurality of activities; and
wherein the first device is adapted to store at least some of the information sensed by the first sensor.

2. The system of claim 1, the system further comprising a second device mounted to a second portion of the body, the second device comprising a second sensor,
wherein the second sensor is adapted to sense information pertaining to the performance of the body in the at least one of a plurality of activities, and
wherein at least one of the first device and the second device is adapted to store at least some of the information sensed by the second sensor.

3. The system of claim 1, the system further comprising a computer, the computer comprising a second computer processor,
wherein the computer is adapted to operate at least one analytics application.

4. The system of claim 2, the system further comprising a computer, the computer comprising a second computer processor,
wherein the computer is adapted to operate at least one analytics application.

5. The system of claim 4, wherein at least one of the first device and the second device is adapted to transfer at least some of the information sensed by at least one of the first sensor and the second sensor to the computer.

6. The system of claim 1, wherein the first sensor is an accelerometer.

7. The system of claim 6, wherein the accelerometer is adapted to sense acceleration along three orthogonal axes.

8. The system of claim 1, wherein the activity is swimming.

9. The system of claim 1, wherein the first computer processor is adapted to infer at least one event pertaining to the performance of the body in at least one of a plurality of activities based on at least some of the information sensed by the first sensor.

10. The system of claim 9, wherein the first computer processor is adapted to automatically infer at least one of the commencement or the conclusion of the at least one event.

11. The system of claim 4, wherein at least one of the first computer processor and the second computer processor is adapted to infer at least one event pertaining to the performance of the body in at least one of a plurality of activities based on at least some of the information sensed by at least one of the first sensor and the second sensor.

12. The system of claim 1, wherein the first device is adapted to configure the state of operation of the system based on at least one manually entered parameter.

13. The system of claim 1, wherein the first device is adapted to automatically configure the state of operation of the system based on at least some of the information sensed by the first sensor.

14. The system of claim 1, wherein the first computer processor is adapted to automatically recognize a specific activity being performed by the body based on at least some of the information sensed by the first sensor.

15. The system of claim 1, wherein the first device further comprises a feedback means, and the first device is adapted to provide feedback to the body via the feedback means.

16. The system of claim 1, wherein the first device is adapted to be mounted to a plurality of portions of the body.

17. A system for monitoring the performance of a swimmer, the system comprising:
a first device mounted to a first portion of the swimmer, the first device comprising a first sensor and a first computer processor,
wherein the first sensor is adapted to sense information pertaining to the performance of the swimmer; and
wherein the first device is adapted to store at least some of the information sensed by the first sensor.

18. The system of claim 17, the system further comprising a computer comprising a second computer processor, wherein the computer is adapted to operate at least one analytics application.

19. The system of claim 17, wherein the first computer processor is adapted for at least one of identifying a stroke, recognizing an event, inferring the commencement of an event, inferring the conclusion of an event, identifying a stroke cycle, and calculating at least one of a distance-per-stroke, a measure of force, a measure of efficiency and a measure of intensity, based on at least some of the information sensed by the first sensor.

20. The system of claim 18, wherein at least one of the first computer processor and the second computer processor is adapted for at least one of identifying a stroke, recognizing an event, inferring the commencement of an event, inferring the conclusion of an event, identifying a stroke cycle, and calculating at least one of a distance-per-stroke, a measure of force, a measure of efficiency and a measure of intensity, based on at least some of the information sensed by the first sensor.

21. The system of claim 17, wherein the first computer processor is adapted to automatically configure the state of operation of the system based on at least some of the information sensed by the first sensor.

22. The system of claim 18, wherein at least one of the first computer processor and the second computer processor is adapted to automatically configure the state of operation of the system based on at least some of the information sensed by the first sensor.

23. The system of claim 17, wherein the first sensor is adapted to sense at least one biomechanical measure.

24. The system of claim 18, the system further comprising a second device mounted to a second portion of the swimmer, the second device comprising a second sensor,
wherein the second sensor is adapted to sense information pertaining to the performance of the swimmer; and
wherein at least one of the first device and the second device is adapted to store at least some of the information sensed by the second sensor.

25. The system of claim 24, the system further comprising a third device mounted to a third portion of the swimmer, the third device comprising a third sensor, wherein the third sensor is adapted to sense a physiological condition of the swimmer.

26. A method for monitoring the performance of an activity by a body, the method comprising the steps of:
providing a first device comprising a first sensor and a computer processor, wherein the first sensor is adapted to sense information;
configuring the first device to sense information during the performance of the activity; and
storing at least some of the information sensed by the first sensor as the activity is performed.

27. The method of claim 26, the method further comprising the steps of:
providing a second device comprising a second sensor, wherein the second sensor is adapted to sense information, wherein the second sensor is adapted to be mounted to a portion of the body;
configuring the second device to sense information during the performance of the activity; and
storing at least a portion of the information sensed by the second sensor as the activity is performed.

28. The method of claim 27, the method further comprising the step of transferring at least some of the information sensed by the first sensor to a computer, wherein the computer comprises a computer processor, and wherein the computer is adapted to operate at least one analytics application.
